(19) United States
(12) Patent Application Publication
(10) Pub. No.: US 2026/0240468 A1
(43) Pub. Date: Aug. 20, 2026

(54) MENSTRUAL FLUID SAMPLING DEVICE

(71) Applicant: IOTA DIAGNOSTIC PRIVATE LIMITED, Gandhinagar (IN)

(72) Inventors: Vaibhav Tatyasaheb Shitole, Gandhinagar (IN); Rajiv Sharma, Gandhinagar (IN); Somesh Chandra, Gandhinagar (IN); Vidisha Septa, Gandhinagar (IN); Pradeep Kumar Verma, Gandhinagar (IN)

(21) Appl. No.: 19/643,754

(22) Filed: Apr. 9, 2026

Related U.S. Application Data
(63) Continuation of application No. PCT/IN2024/051996, filed on Oct. 7, 2024.

(30) Foreign Application Priority Data
Oct. 9, 2023 (IN) 202321067654

(51) Int. Cl. A61B 5/15

(52) U.S. Cl. CPC A61B 5/150045; A61B 5/150358

(57) ABSTRACT

A menstrual fluid sampling device comprising: a strip(S) made with a super-macroporous material, which collects and stores bio-fluids for analysis, said material being formed into blobs (B) and placed atop said strip(S), in a discrete manner such that two adjacent blobs (B) are spaced apart from each other, each of said blobs (B) serve as receptacle material for biological fluids, said blobs (B) sandwiched in between two membrane layers (UL, LL); said two membrane layers (UL, LL) consisting, essentially, of an upper layer (UL) having: one or more chambers (CH), each of said blobs being stored in corresponding chambers (CH) by way of encapsulation; perforations, with openings (O), which allow fluid inside said one or mode chambers (CH) by capillary action; and said two membrane layers (UL, LL) consisting, essentially, of a lower layer (LL) having: a removable layer configured to be peeled off before use.

### RELATED APPLICATIONS

This application claims priority under 35 U.S.C. § 120 to, and is a continuation of, co-pending International Application PCT/IN2024/051996, filed Oct. 7, 2024 and designating the US, which claims priority to IN application No. 202321067654, filed Oct. 9, 2023, such IN application also being claimed priority to under 35 U.S.C. § 119. These IN and International applications are incorporated by reference herein in their entireties.

### FIELD

This invention relates to the field of biomedical engineering. Particularly, this invention relates to the field of diagnostics. Specifically, this invention relates to a menstrual fluid sampling device, aiming to facilitate diagnostic and health assessments of menstruating women.

### BACKGROUND

Healthcare plays a vital role for maintaining good health, preventing illnesses, and managing existing conditions effectively. Regular check-ups, preventive care, and timely treatment contribute to improved overall well-being and a higher quality of life.

However, for diagnosis of a few infectious diseases, disease biomarkers, and others, bio-analyte presents several challenges and problems when a bio-specimen needs to be sampled from female subjects; typically, from lower abdominals areas, when it comes to reproductive health and infectious diseases diagnosis.

Mostly, according to available procedures of the prior art, there includes pap smear, liquid-based cytology, endocervical curettage, colposcopy-directed biopsy, and the like procedures. Nevertheless, these practices are very painful and cumbersome towards patients because of cells/tissue sample obtained through a small surgery with special tools and techniques.

Also, this process needs healthcare professionals to collect the bio-specimen for diagnosis requirement and necessitates a sophisticated environment and setup.

Furthermore, societal stigma and cultural beliefs surrounding reproductive health and sexuality may discourage women from seeking regular screenings and diagnosis or discussing symptoms related to cervical related disease.

Menstrual fluid is a complex biological fluid comprising blood, mucus and endometrial tissue from the uterine lining. Numerous research studies have substantiated menstrual fluid as a valuable specimen for disease diagnosis, primarily due to the presence of diverse biomarkers and other bio-analytes derived from the blood, mucus, infected basal epithelia cells or endometrial tissues. Furthermore, menstrual fluid serves as a rich source of stem cells, which can be easily isolated non-invasively. The isolated stem cells exhibit high proliferation and multi-lineage differentiation potency. Thus, menstrual fluid emerges as an excellent alternative as a bio-specimen for disease screening, diagnosis, and a potential source for tissue engineering applications.

### SUMMARY

To address the current issues related to sample collection, there is a need for a device designed to collect menstrual fluids from females during their menstrual periods, which can be utilized for diagnostic, research, and medical applications.

An object of the invention is to provide a device designed to collect menstrual fluids from females during their menstrual cycles, and preserve it in a dried matrix form, which can be utilized for diagnostic, research, and medical applications.

Another object of the invention is to provide a device, being a non-invasive device, for easier collection of the menstrual fluids, remotely, at patient's privacy for diagnostic and medical requirements.

Another object of the invention is to provide a self-sampling device for women's health assessment and disease diagnosis based on menstrual fluid.

Another object of the invention is to provide easier sample storage, transportation, and laboratory processing for downstream and upstream diagnostic, research and medical application.

Another object of the invention is to provide easier sample exposure and enable the detection of antigens or biomarkers in a sample through colorimetric reaction or an antigen-antibody reaction.

According to this invention, there is provided a menstrual fluid sampling device. This invention pertains to an improved approach for collecting menstrual samples, passively, from females, specifically for diagnostic purposes and various medical applications. The proposed solution is a non-invasive method of collecting menstrual fluid samples, which could be very affordable as compared to current available screening and diagnostic options. The device is designed in a manner for easier collection of the menstrual fluids, remotely, at patient's privacy for diagnostic and medical requirements. The device comprises a special proprietary material, which collects and stores menstrual bio-fluid for analysis. The material is designed in a manner to collect a predefined volume of fluid, thus it could be considered for downstream or upstream applications such as quantifications of bio-analyte and tissue engineering, respectively.

According to this invention, there is provided a menstrual fluid sampling device comprising:
a strip made with a super-macroporous material, which collects and stores bio-fluids for analysis, said material being formed into blobs and placed atop said strip, in a discrete manner such that two adjacent blobs are spaced apart from each other, each of said blobs serve as receptacle material for biological fluids, said blobs sandwiched in between two membrane layers;
   said two membrane layers consisting, essentially, of an upper layer having:
      one or more chambers, each of said blobs being stored in corresponding chambers by way of encapsulation;
      perforations, with openings, which allow fluid inside said one or more chambers by capillary action; and
   said two membrane layers consisting, essentially, of a lower layer having:
a removable layer configured to be peeled off before use.

In at least an embodiment, said device comprising a matrix formed by said two membrane layers and said blobs.

In at least an embodiment, said blobs being hemispherical blobs with their bases being supported by said lower layer.

In at least an embodiment, said strip comprising a linearly, axially, extending narrow extension for handling said strip after collecting said sample.

In at least an embodiment, blobs being encapsulated under said corresponding openings, located on an operative top of each of the chambers of the upper layer, such that fluid from a body reaches said openings and into said blobs underneath.

In at least an embodiment, said super-macroporous material being a hydrophilic super-macroporous material having an interconnected porous network capable to:
absorb biological fluid through capillary action; and
hold absorbed biological fluid within the porous network.

In at least an embodiment, said super-macroporous material being a hydrophilic super-macroporous material having pore size ranging from 10-120 μM with porosity of the material ranging from 90-95%.

In at least an embodiment, said super-macroporous material being coated with antimicrobial agents selected from a group consisting of Chlorine Compounds (Chlorine dioxide or sodium hypochlorite), Silver nanoparticles, benzalkonium chloride, and Triclosan.

In at least an embodiment, said super-macroporous material being enzymatically treated with enzymes selected from a group consisting of DNase inhibitors, RNase Inhibitors, Protease Inhibitors.

In at least one embodiment, the super-macroporous material is treated with antibodies and selected reagents to form antigen-antibody reactions and colorimetric reactions, enabling detection of disease biomarkers and pathogens.

In at least an embodiment, the strip contains a plurality of super-macroporous materials, exhibiting similar or varied chemical compositions achieved through chemical modification using chemicals, antibodies, and reagents in order to achieve a desired outcome.

### BRIEF DESCRIPTION OF THE ACCOMPANYING DRAWINGS

The invention will now be described in relation to the accompanying drawings, in which:

FIG. 1 illustrates a the entire device;

FIG. 2 illustrates a top view of a strip;

FIG. 3 illustrates an exploded view of a strip;

FIG. 4 illustrates a cross sectional view of the strip showing the porous material arrangement inside the membranes;

FIGS. 5a, 5b, 5c illustrate top views of the device;

FIGS. 6a, 6b, 6c illustrate side views of the device;

FIG. 7 illustrates a strip handling within a cartridge for laboratory sample analysis;

FIG. 8A illustrates SEM images of the supermacroporous material;

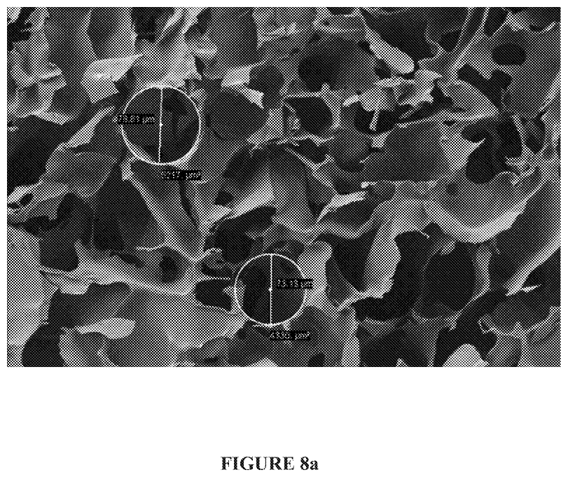

FIG. 8B illustrates the supermacroporous material after exposure to blood, showing deposition of tissues and/or cells within the pores; and

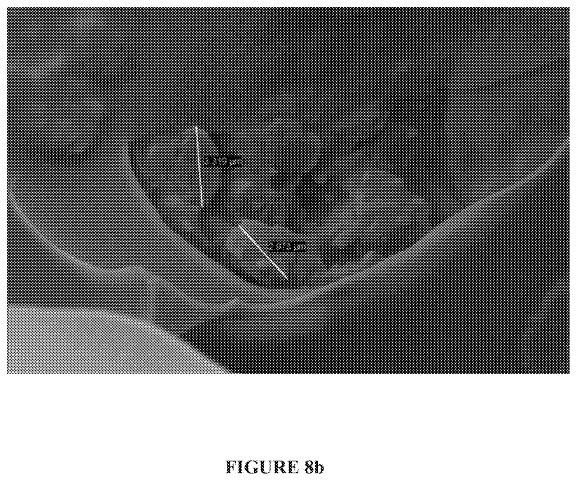

FIG. 8C illustrates a cross-sectional view of the material, demonstrating uniform porous architecture throughout the structure.

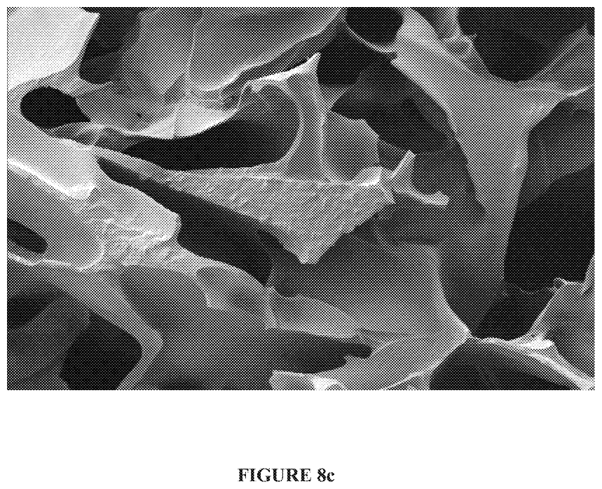

### DETAILED DESCRIPTION

According to this invention, there is provided a menstrual fluid sampling device.

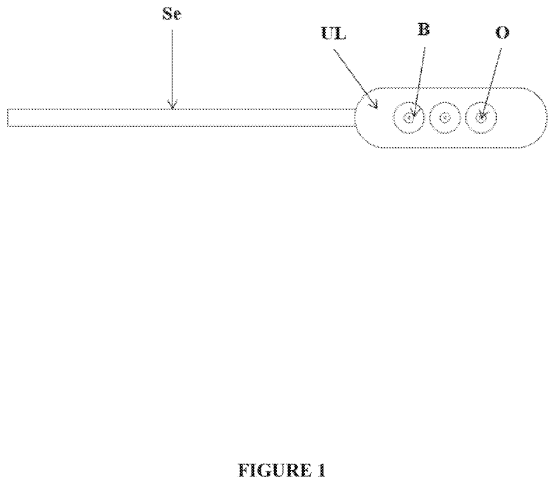

FIG. 1 illustrates a the entire device;

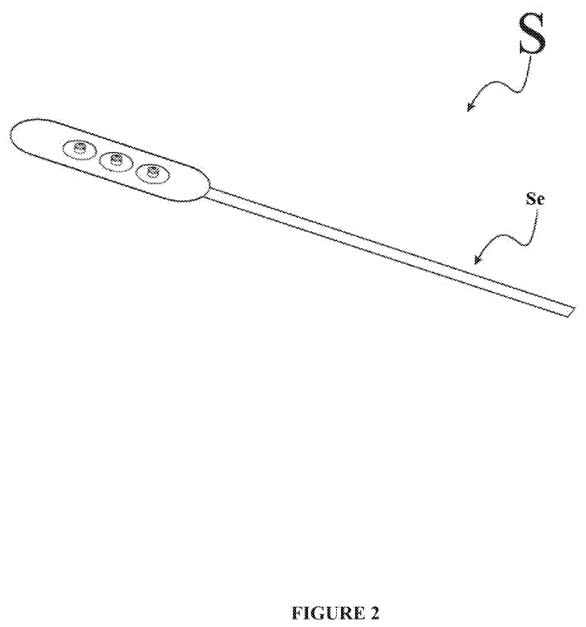

FIG. 2 illustrates a top view of a strip;

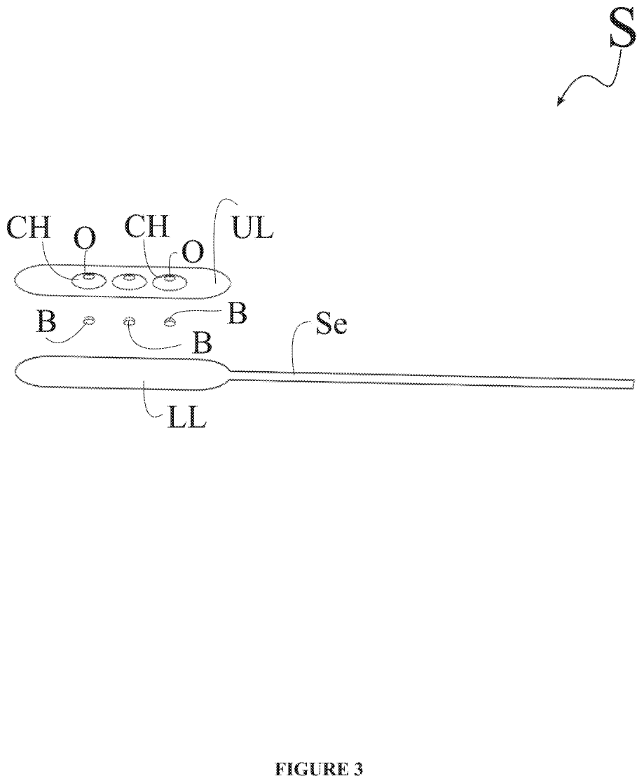

FIG. 3 illustrates an exploded view of a strip;

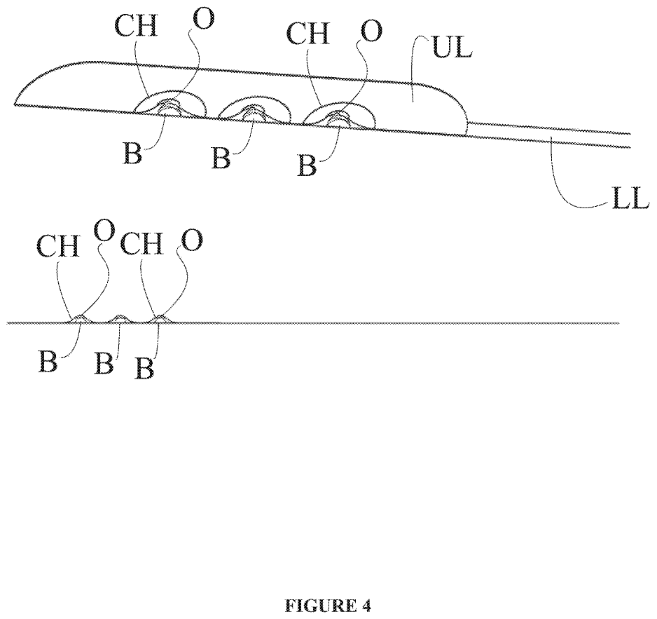

FIG. 4 illustrates a cross sectional view of the strip showing the porous material arrangement inside the membranes;

In at least an embodiment, there is provided a strip(S) made with a super-macroporous material, which collects and stores menstrual bio-fluid for analysis. The material is hydrophilic in nature having interconnected porous network capable to:
absorb the biological fluid through capillary action; and
hold absorbed biological fluid within the porous network.

### Porous Material Description:

| | 30 μL | | 50 μL | | 100 μL | |
|---|---|---|---|---|---|---|
| Height | 1.6 ± 0.2 | mm | 2.3 ± 0.2 | mm | 3.5 ± 0.2 | mm |
| Volume | 19 ± 2 | mm³ | 32 ± 2 | mm³ | 52 ± 2 | mm³ |
| Weight (mg) | 1.8 ± 0.2 | mg | 3.0 ± 0.7 | mg | 6.5 ± 0.7 | mg |
| Porosity % | 94 ± 2% | | | | | |
| Swelling Index | 15 ± 2 | | | | | |
| Blood collection Time, | 2-3 | seconds | 4-6 | seconds | 6-8 | seconds |

when contacted with
enough blood pool

Pore size of the material is from 10-120 μM; thus, it can easily absorb and store blood, endometrial cells, vaginal fluid, and any other complex biological fluids. Porosity of material is in the range of 90-95%. The material can be treated with certain chemical and agents, for desired results, such as antimicrobials, stabilizers, enzymes and antibodies. In other words, the blobs (B) need to be removed from the encapsulant of the strip(S) through cutting along the upper membrane (UL) and lower membrane (LL); thus, the blob (B) can be utilized for laboratory analysis. etc. The porous materials can be detached from the strip when analysis is required. The material is designed in a manner to collect a predefined volume of fluid range from 10 μL to 1 ml precisely with different size of porous material variant; thus, the collected bio-specimen shall be used for qualitative and quantification of bio-markers, viral loads and proteins, etc. This material is formed into blobs (B) so that they serve as receptacle material for menstrual fluids, and these are sandwiched in between two layers (UL, LL) of membranes (typically, plastic membrane). Typically, this strip(S) has, atop it, discretely placed, one or more blobs (B) of porous proprietary material (hydroporous material) which is predefined for volume collection and sandwiched in between the two membranes (UL, LL) of the material. Each blob (b) porous material is stored within a separate chamber (CH), formed in the upper layer (UL), by way of encapsulation. The position and arrangement of the blobs (B) porous materials in strip is kept in a manner such that it could easily be exposed to the menstrual flow through openings (O) on an operative top of each of the chambers (CH) of the upper layer (UL). Collection will occur simultaneously across all blobs (B), facilitated by individual openings (O), for each blob (B), to capture the fluid/biospecimen. The strip is extended with a narrow extension (Se), intend to easily remove the strip after menstruation without contamination by an operator.

The supermacroporous material is characterised by its functional properties rather than by any particular chemical composition. The supermacroporous material suitable for use in the present invention is any hydrophilic material having an interconnected porous network that satisfies the following functional parameters, as disclosed herein: a pore size ranging from 10 μm to 120 μm; a porosity in the range of 90-95%; a swelling index of 15±2; and an absorption capacity enabling collection of a predefined volume of biological fluid in the range of 10 μL to 1 mL, depending on the size of the blob (B). In a non-limiting exemplary embodiment, the material absorbs 50±5 μL of blood within 15 to 30 seconds of blood pooling, irrespective of the blood's viscosity and the arrangement of the blobs (B) within the device. Any hydrophilic material having an interconnected porous network and satisfying the aforesaid functional parameters falls within the scope of the present invention, and the invention is not limited to any particular chemical composition of the supermacroporous material.

The two layers (UL, LL) and the blobs (B) of the hydroporous material, therebetween, forms a matrix of this invention.

In at least an embodiment, an upper layer (UL), of the two layers, is perforated with small openings (O), which allow fluid to flow inside the chamber (CH) of membrane. Thus, the material gets exposes and collects the menstrual fluid inside the material by capillary action. The upper layer is directed towards vaginal openings.

In at least an embodiment, a lower layer (LL), of the two layers, allows integration with a menstrual pad. This lower layer (LL) is configured to be, partially, adhered with a menstrual pad and has an extension to easily peel off/remove and handle the entire strip matrix for further downstream or upstream applications and to avoid cross contamination and biohazard while handling the sampled matrix.

The developed device collects menstrual blood, in the blobs (B), within a few minutes after its discharge, and stores it into the supermacro porous material in the device.

The openings (O) of the upper layer (UL) are characterised by their function rather than by any particular dimension. The openings (O) are sized and configured to permit entry of menstrual fluid, comprising blood, mucus, infected basal epithelial cells, and endometrial tissue components, into the chamber (CH) by capillary action, such that the supermacroporous material of the blob (B) thereunder is brought into fluid contact with the menstrual fluid. In a non-limiting exemplary embodiment, the openings (O) are sized such that the whole blood absorption process by the supermacroporous material is completed within 15 to 30 seconds after sufficient blood pooling at the upper layer (UL), as confirmed by the experimental results described herein. Further, the openings (O) are configured such that collection occurs simultaneously across all blobs (B), each blob (B) being served by its individual opening (O). The invention is not limited to any particular shape or dimension of the openings (O), provided that the openings (O) are configured to permit capillary-driven entry of menstrual fluid into the chamber (CH) and into the supermacroporous material of the blob (B) thereunder.

The predefined volume collection capability of the supermacroporous material is a function of its geometry, porosity, and the hydrophobic character of the upper layer (UL). Each blob (B), being hemispherical in shape with its base supported by the lower layer (LL), defines a fixed geometric volume. The interconnected porous network of the blob (B), having porosity in the range of 90-95%, absorbs biological fluid up to the saturation capacity dictated by said fixed geometric volume. Once the blob (B) reaches saturation, excess menstrual fluid accumulates on the hydrophobic top surface of the upper layer (UL) and does not enter the blob (B), thereby preventing over-absorption. This mechanism enables each blob (B) to collect a predefined volume of menstrual fluid irrespective of the viscosity or flow rate of the menstrual fluid, as confirmed by gravimetric analysis wherein all matrices consistently absorbed 50±5 μL of blood irrespective of the blood's viscosity and the arrangement of materials within the devices.

In terms of treatment of the supermacro porous material, in preferred embodiment, can undergo treatment with various chemicals and reagents to enable storage and stabilization of biospecimens/fluids for various medical and omics applications.

According to a non-limiting exemplary embodiment, in order to obtain antimicrobial nature in the porous material, this can be achieved by material treatment or coating with the antimicrobial agents such as Chlorine Compounds (Chlorine dioxide or sodium hypochlorite), Silver nanoparticles, benzalkonium chloride, Triclosan, and the like.

According to a non-limiting exemplary embodiment, enzyme treatment can be done in order to stabilize Nucleic acid, inactivation of nucleases and biochemical reaction to enhance sample recovery and quality for analytical outcome such as DNase inhibitors, RNase Inhibitors, Protease Inhibitors, and the like.

In at least an embodiment, the invention comprises a cartridge for storage and shipment purposes. It is specifically designed to facilitate the storage and transportation of the sampled matrix to a laboratory or other desired locations for diagnostic and medical applications. The cartridge is completely sealed and air tight to prevent any environmental biohazard, and its design ensures that the matrix remains centred and does not adhere to the walls of the cartridge. The cartridge's design facilitates insertion of strip edges longitudinally running through guiding rails, ensuring the samples remain centered. This arrangement effectively prevents the samples from contacting the cartridge walls, thereby avoiding damage and wear. Moreover, the cartridge provides ample space for desiccants, which effectively maintain the material in a dry state for long-term preservation, specifically intended for research, bio-banking purposes. The cartridge is designed for the storage and transportation of sampled strip without contaminating environment and other specimens. The design keeps the strip centred to avoid sample contamination with the walls of cartridge. Typically, the porous collection matrix, in strip, is a 3D dimensional porous material that is in a geometric shape, capable to collect precise volume irrespective of nature of menstrual fluid. Naturally material is in the hemisphere in shape, base is positioned on the lower layer (LL). There are multiple hemisphere-shaped porous materials arranged in the strip, having similar dimensions and characteristics. The strip may also contain plural porous materials, exhibiting similar or varied chemical compositions achieved through chemical modification using specific chemicals and reagents, to achieve a desired outcome. These materials can be conveniently—removed and placed either within a laboratory tube or in the wells of an extraction plate manually or through automated setup. This allows for the easy retrieval of samples from the porous materials for both downstream and upstream applications. The bio-analytes from the porous matrix are easily retrievable in organic, inorganic solvents and laboratory extraction media and can be utilized for further analysis. After obtaining the extract the genetic material, proteins, drugs, bio-analyte etc will be processed further with available standard laboratory assay methods for analysis or medical use. The porous material can be pretreated with certain chemicals and reagents to obtain desired interest of applications.

The device is, specifically, a kit which includes a strip and cartridge and is specifically designed to easier collection, storage, transportation and analysis of menstrual fluid for diagnostic and medical applications.

The following steps disclose a procedure of sampling using this invention:
1) Wash hands thoroughly with soap and water before handling the kit to maintain hygiene during the sample collection
2) Install the strip with a menstrual pad carefully at its centre by removing its adhesive backing and gently pressing an edge of the strip over the menstrual pad;
3) Ensure that the adhesive sticks firmly to the menstrual pad;
4) Place the pad inside underwear;
5) Ensure that the perforated opening side faces vaginal opening—the pad should be centered and positioned to catch menstrual flow;
6) Expose the menstrual pad for few hours during menstruation, until it absorbs the menstrual flow in the strip;
7) Remove the pad from underwear, use the provided outside extension to peel off or remove the strip, ensuring hygiene and preventing cross-contamination;
8) Store the strip into the provided cartridge, and then hand it over to medical personnel for diagnosis, research and medical use.

Following steps discloses a procedure for sample extraction using this invention.
1) The super-macroporous material need to remove manually from the strip;
2) The material needs to immerse into the respective volume of distilled water or extraction media, and allow it stand at room temperature or incubator at least 15-20 minutes;
3) Later add 3-5-fold extraction media and stand it for at least 15-20 minutes at room temperature or incubator;
4) Later sample will be subject to nucleic acid extraction, drug extraction, protein extraction, and biochemical analysis as per the available standard laboratory assay methods.

This device utilizes menstrual blood for diagnosis and research applications. Menstrual blood contains numerous biomarkers, cells, proteins, and enzymes that can aid in early diagnosis as well as for the tissue engineering applications.

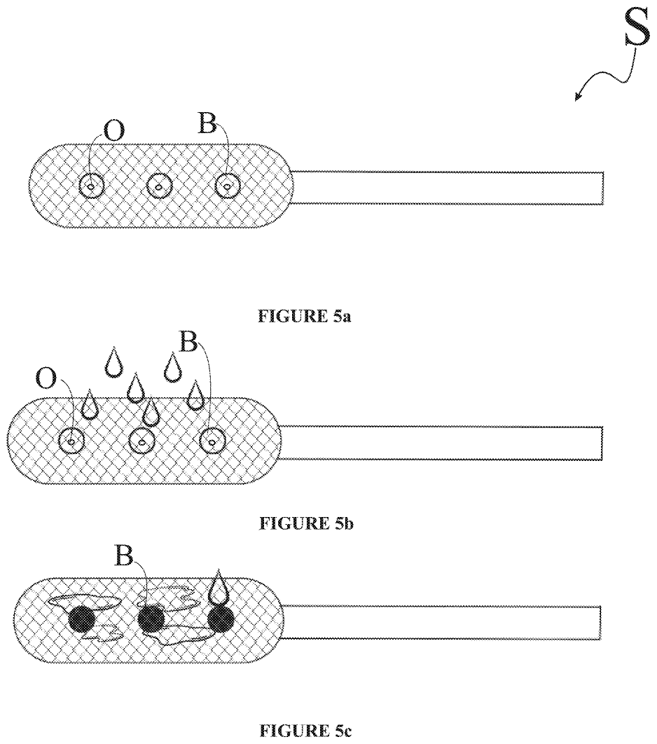

FIGS. 5a, 5b, 5c illustrate a diagrammatic view of device collecting blood within a porous material.

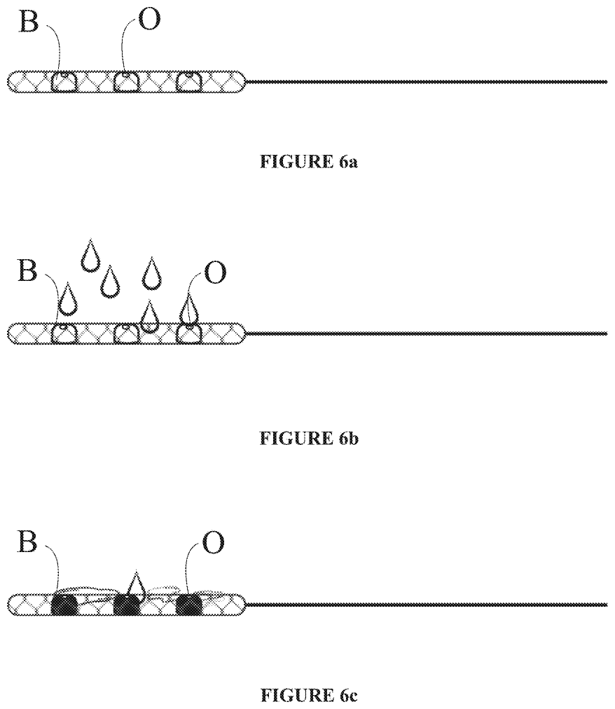

FIGS. 6a, 6b, 6c illustrate diagrammatic side views of the device collecting blood within the porous materials

In FIG. 5b and FIG. 6b, it is shown that blood is discharged from menstruation onto the device.

In FIG. 5c and FIG. 6c, it is shown that after wearing for some time, the super macroporous material of the device becomes fully red and saturated, with excess blood accumulating on its top surface of upper membrane.

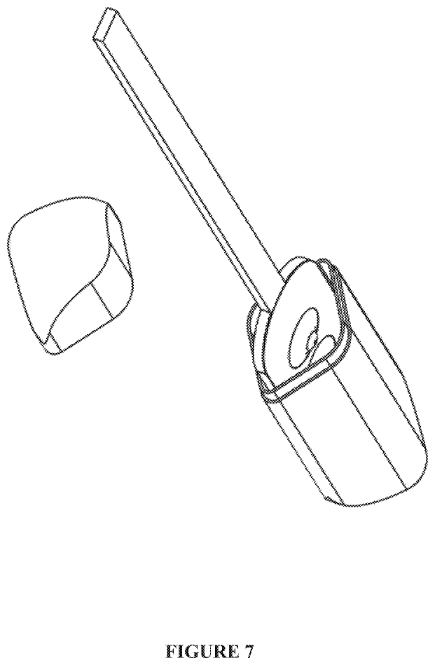

FIG. 7 illustrates a strip handling within a cartridge for laboratory sample analysis.

According to a non-limiting exemplary embodiment, device design performance was evaluated through a simulated laboratory experiment utilizing human blood. The invented device was exposed to simulated menstrual flow in laboratory conditions with human blood to evaluate its performance and effectiveness in menstrual blood collection and storage in the porous matrix for subsequent laboratory analysis. The objective was to:
1) Design performance assessment of M-Strip device for menstrual blood collection and storage; and
2) Volumetric Measurement of sampled blood through gravimetric analysis.

### Methodology:

Five devices were exposed to sequential blood loading through a micropipette to simulate menstrual blood flow exposure. Each device underwent two blood loading events through the micropipette, with a 2-minute interval between them. The blood was discharged on the upper membrane of each experimental M-strip Device. Following the first blood discharge, most porous matrices became fully saturated, as indicated by the complete expansion and fully red-colored material. The volume of blood collected was assessed using gravimetric analysis. Before exposure, each individual porous material was weighed and assembled into the device. The weight of the saturated material after 2nd blood loading was then measured using a laboratory weighing balance to determine the volume of blood absorption, based on the gravimetric method for volume analysis.

The following observation table was plotted:

| Device/ | | Blood Volume | Matrix Fully |
|---|---|---|---|
| M-Strip | Blood Discharge (μL) | Collection (μL) | Absorption |
| Device 1 | | | |
| A1 | 1^{st} Exposure: 200 μL | 49 | T1 |
| A2 | 2^{nd} Exposure: 200 μL | 48 | T1 |
| A3 | | 48 | T1 |
| Device 2 | | | |
| A1 | 1^{st} Exposure: 200 μL | 50 | T1 |
| A2 | 2^{nd} Exposure: 200 μL | 53 | T1 |
| A3 | | 47 | T1 |
| Device 3 | | | |
| A1 | 1^{st} Exposure: 200 μL | 51 | T1 |
| A2 | 2^{nd} Exposure: 200 μL | 53 | T1 |
| A3 | | 47 | T1 |
| Device 4 | | | |
| A1 | 1^{st} Exposure: 200 μL | 49 | T2 |
| A2 | 2^{nd} Exposure: 200 μL | 47 | T2 |
| A3 | | 47 | T1 |
| Device 5 | | | |
| A1 | 1^{st} Exposure: 200 μL | 52 | T1 |
| A2 | 2^{nd} Exposure: 200 μL | 53 | T2 |
| A3 | | 51 | T2 |

A1, A2 and A3 denotes embedded porous material in the M-strip Device.

T1—indicates the porous material achieved complete absorption after the 1st blood loading.

T2—indicates the porous material achieved complete absorption after the 2nd blood loading.

All devices exhibited complete absorption of discharged blood, following the 2nd blood loading. This porous material collection was facilitated by capillary mechanisms of small openings on the upper membrane and the supermacroporous hydrophilic nature of the porous material. However, excess blood was observed on the upper membrane of the M-strip that indicates porous material will predominantly absorb blood during the menstrual flow. The whole blood absorption process occurred within 15 to 30 seconds after enough blood pooling. Across all matrices within the devices consistently absorbed of 50±5 μL of blood, irrespective of the blood's viscosity and the arrangement of materials within the devices.

Additionally, according to a non-limiting exemplary embodiment, the supermacroporous material is characterized to evaluate its capacity for absorption of complex biological specimens. Menstrual blood represents a heterogeneous fluid comprising blood, mucus, infected basal epithelial cells, and/or endometrial tissue components. The size of such biological constituents may range from nanometer to micrometer scale.

In an embodiment, the supermacroporous material is characterized using scanning electron microscopy (SEM) analysis. The SEM analysis demonstrates that the material exhibits a highly porous architecture, with pore sizes ranging from approximately 10 μm to 120 μm.

The pore size range of 10 μm to 120 μm of the supermacroporous material is selected to correspond to the size range of biological constituents present in menstrual fluid, which, as noted herein, may range from nanometer to micrometer scale. The interconnected porous network of the supermacroporous material thus facilitates entry and retention of blood, mucus, infected basal epithelial cells, and endometrial tissue components within the pore network via capillary action. This correspondence between pore size and constituent size enables the material to function as a specimen receptacle for the complete heterogeneous composition of menstrual fluid, suitable for downstream diagnostic and biomedical applications.

Owing to this porous structure, the material facilitates efficient absorption of fluid, along with associated tissues and cellular components, via capillary action, enabling deposition within the pore network. In this manner, the material functions as a specimen receptacle configured to collect, store, and preserve biofluids in a dried state, suitable for subsequent diagnostic and biomedical applications.

FIG. 11A illustrates SEM images of the supermacroporous material.

FIG. 11B illustrates the supermacroporous material after exposure to blood, showing deposition of tissues and/or cells within the pores.

FIG. 11C illustrates a cross-sectional view of the material, demonstrating uniform porous architecture throughout the structure.

Additionally, according to a non-limiting exemplary embodiment, the device is evaluated in menstruating female subjects to assess clinical performance. The strip is utilized in accordance with the instructions and procedures described herein, and the collected sample is subsequently transported to a laboratory for analysis.

In an embodiment, the sample collected on the strip is subjected to nucleic acid extraction using standard extraction media and established protocols. The extracted nucleic acids are then processed using polymerase chain reaction (PCR) assays for detection of human papillomavirus (HPV) genotypes.

In a comparative evaluation, clinical samples collected using the strip are analyzed alongside samples obtained using a gold-standard cervical swab collection method performed by a medical operator. The results demonstrate 100% concordance between menstrual blood samples collected via the strip and cervical swab samples for HPV detection using PCR testing.

In this exemplary embodiment, the sample collected on the strip is processed in accordance with the sample extraction procedure described herein, wherein the supermacroporous material is removed from the strip, immersed in extraction media, and subjected to nucleic acid extraction. The extracted nucleic acids are subsequently analysed using PCR assays for detection of HPV genotypes. The cervical swab samples, collected in parallel by a medical operator using a gold-standard collection method, are subjected to the same nucleic acid extraction and PCR testing protocol. The 100% concordance observed between the two collection methods demonstrates that the strip, used non-invasively by the subject in accordance with the sampling procedure described herein, yields nucleic acid of sufficient quality and quantity for PCR-based detection of HPV genotypes, confirming the clinical utility of the device for molecular diagnostic applications.

### Technical Advantages

Non-invasive and painless sample collection using menstrual biofluid;
User-friendly and easy to handle device;
No need for specialized tools or techniques;
No requirement for healthcare professionals to collect bio-specimen;
No need for a sophisticated environment and setup for sample collection.

The TECHNICAL ADVANCEMENT of this invention lies in providing a the bio-sampling kit with a strip, of this invention, which allows for controlled gathering of menstrual sample when used with a menstrual pad; the strip is such that it is capable to collect precise volume irrespective of nature of menstrual fluid. Along with its cartridge, the kit provides hazard-free and contamination-free storage and transportation. This kit allows the end-user to conveniently collect their own samples at home; which was, hitherto, not possible.

While this detailed description has disclosed certain specific embodiments for illustrative purposes, various modifications will be apparent to those skilled in the art which do not constitute departures from the spirit and scope of the invention as defined in the following claims, and it is to be distinctly understood that the foregoing descriptive matter is to be interpreted merely as illustrative of the invention and not as a limitation.

1. A menstrual fluid sampling device comprising:
a strip;
blobs of a super-macroporous material configured to collect and store a biofluid for analysis, wherein the blobs are on a top of the strip and spaced apart from each other, wherein the blobs are configured to receive the biofluid;
an upper membrane layer having chambers, wherein each chamber of the chambers encapsulates a blob of the blobs, wherein the upper membrane includes perforations that admit fluid into the chambers by capillary action; and
a lower membrane layer having a removable layer configured to be peeled off before use.

2. The device of claim 1, wherein the two membrane layers and the blobs for a matrix.

3. The device of claim 1, wherein the blobs are hemispherical blobs with bases supported by the lower membrane layer.

4. The device of claim 1, wherein the strip includes an extension that is narrower than the remainder of the extension for handling the strip after collecting the biofluid.

5. The device of claim 1, wherein the blobs are under the perforations, and wherein the perforations are on a top of the chambers so as to permit the biofluid to pass through the openings and into the blobs underneath.

6. The device of claim 1, wherein the super-macroporous material is a hydrophilic super-macroporous material having an interconnected porous network, wherein the interconnected porous network is configured to absorb the biofluid through capillary action and hold the absorbed biofluid within the interconnected porous network.

7. The device of claim 1, wherein the super-macroporous material is a hydrophilic super-macroporous material having pore size ranging from about 10 to 120 μM and a porosity of about 90 to 95%.

8. The device of claim 1, wherein the super-macroporous material is coated with antimicrobial agents including at least one of a chlorine compound, silver nanoparticles, benzalkonium chloride, and triclosan.

9. The device of claim 1, wherein the super-macroporous material is coated with chlorine dioxide and/or sodium hypochlorite.

10. The device of claim 1, wherein the super-macroporous material is enzymatically treated with enzymes including at least one of a DNase inhibitor, a RNase Inhibitor, and a Protease Inhibitor.

11. The device of claim 1, further comprising:
blobs of another super-macroporous material different from the super-macroporous material.

12. The device of claim 1, wherein the super-macroporous material includes antibodies and/or reagents configured to form antigen-antibody reactions and/or colorimetric reactions when exposed to the biofluid so as to indicate disease biomarkers and/or pathogens.
